(19) United States
(12) Patent Application Publication
(10) Pub. No.: US 2017/0224205 A1
(43) Pub. Date: Aug. 10, 2017

(54) LIGHT ENDOSCOPE SYSTEM FOR IMAGING, LIGHT DELIVERY, AND THERAPY RESPONSE MONITORING

(71) Applicants: Ulas Sunar, Beavercreek, OH (US); Dan Rohrbach, Beavercreek, OH (US); Jeremy Kress, Beavercreek, OH (US)

(72) Inventors: Ulas Sunar, Beavercreek, OH (US); Dan Rohrbach, Beavercreek, OH (US); Jeremy Kress, Beavercreek, OH (US)

(73) Assignee: Wright State University, Dayton, OH (US)

(21) Appl. No.: 15/425,822

(22) Filed: Feb. 6, 2017

Related U.S. Application Data
(60) Provisional application No. 62/291,261, filed on Feb. 4, 2016.

(51) Int. Cl. A61B 1/05; A61N 5/06; A61B 1/055; A61B 1/018; A61B 1/06; A61B 1/04

(52) U.S. Cl. CPC A61B 1/051; A61B 1/0638; A61B 1/0684; A61B 1/043; A61B 1/055; A61B 1/018; A61N 5/062; A61N 5/0601; A61N 2005/0612; A61N 2005/0626

(57) ABSTRACT

An endoscopic imaging system is disclosed that includes an endoscope having a first channel and a second channel, a high-power, multi-wavelength LED array, a digital micro-mirror device that receives light directed from the high-power, multi-wavelength LED array and generates spatial frequency patterns, and dichroic mirror that separates reflectance and fluorescence images.

### CROSS-REFERENCE TO RELATED APPLICATIONS

This application claims the benefit of U.S. Provisional Patent application Ser. No. 62/291,261 entitled “LIGHT ENDOSCOPE SYSTEM FOR IMAGING, LIGHT DELIVERY, AND THERAPY RESPONSE MONITORING” and filed Feb. 4, 2016. The entirety of the above-noted application is incorporated by reference herein.

### BACKGROUND

For optimal phototherapy, there needs to be sufficient light and drug dose present in the target cells for complete cell killing along with sufficient oxygen. Light dose distribution is affected by the local optical parameters (i.e. absorption and scattering) at a therapeutic wavelength, and tissue oxygenation is affected by the vascular parameters such as blood oxygen saturation. Thus, it is desirable to quantify these parameters for accurate light dosimetry and phototherapy monitoring.

During phototherapy treatment field planning, micronodules may not be clinically evident. Drugs (e.g., porphyrins used as photosensitizers in photodynamic therapy or doxorubicin used in chemotherapy) fluoresce strongly and fluorescence signal can provide improved tumor demarcation for accurate light illumination. This allows optimal targeting of tumor cells while preserving the surrounding normal tissue. Thus, an instrument that can provide high contrast for improved tumor demarcation is desired. Raw fluorescence signals, however, are distorted due to strong tissue absorption and scattering, hiding the true fluorescence contrast. Thus, an attenuation corrected quantitative fluorescence imaging system is needed.

Since there is great heterogeneity in optical parameters and drug accumulation as well as tumor size and shape in patients, the light dose needs to be individualized by an instrument that can deliver adaptive light beam shape and intensity on a patient by patient basis.

### SUMMARY

The following presents a simplified summary in order to provide a basic understanding of some aspects of the innovation. This summary is not an extensive overview of the innovation. It is not intended to identify key/critical elements or to delineate the scope of the innovation. Its sole purpose is to present some concepts of the innovation in a simplified form as a prelude to the more detailed description that is presented later.

In one aspect of the innovation, an endoscopic imaging system is disclosed that includes an endoscope having a first channel and a second channel, a high-power, multi-wavelength light emitting diode (LED) array, a digital micro-mirror device that receives light directed from the high-power, multi-wavelength LED array and generates spatial frequency patterns, and dichroic mirror that separates collected reflectance and fluorescence images.

In another aspect of the innovation, a method of phototherapy is disclosed that includes inserting an endoscope into an internal cavity, projecting spatial frequency patterns through a first channel of the endoscope onto tissue, collecting reflected light through a second channel of the endoscope, relaying the reflected light to a pair of cameras, measuring reflectance in one camera and fluorescence in the other camera, analyzing the reflectance and fluorescence images, extracting absorption and scattering properties of the tissue, and adjusting treatment light intensity and shape based on the absorption and scattering properties.

To accomplish the foregoing and related ends, certain illustrative aspects of the innovation are described herein in connection with the following description and the annexed drawings. These aspects are indicative, however, of but a few of the various ways in which the principles of the innovation can be employed and the subject innovation is intended to include all such aspects and their equivalents. Other advantages and novel features of the innovation will become apparent from the following detailed description of the innovation when considered in conjunction with the drawings.

### BRIEF DESCRIPTION OF THE DRAWINGS

FIG. 1a illustrates a combined imaging and treatment system in accordance with an aspect of the innovation.

FIG. 1b is a representative diagram showing light beam shaping and intensity delivering according to target disease shape in accordance with an aspect of the innovation.

FIG. 2 is fiber endoscope with projector input and dual camera detection in accordance with an aspect of the innovation.

FIG. 3a is an illustration of a laparoscope design with dual-channel rod lens modulator with a prism for projection, CCDs for concurrent reflectance and fluorescence imaging, and a beam splitter detection in accordance with an aspect of the innovation.

FIG. 3b is a close-up of the custom dual-rod tip in accordance with an aspect of the innovation.

FIG. 4 illustrates digital micromirror change between 4a. solid pattern illumination, and 4b. shape specific pattern illumination for the procedure in imaging mode and in treatment mode in accordance with an aspect of the innovation.

FIG. 5 illustrates an optical property corrected fluorescence is linear with doxorubicin concentration in accordance with an aspect of the innovation.

FIG. 6 illustrates a system showing that light can be delivered in any shape and different intensity according to the disease location and shape in accordance with an aspect of the innovation.

FIGS. 7a and 7b illustrate an optimized light illumination projection in accordance with an aspect of the innovation.

FIG. 8 is a block diagram illustrating a method of performing phototherapy in accordance with an aspect of the innovation.

FIGS. 9a-9d illustrate the effect of drug release by the endoscope system. Specifically, they contrast absorption of drugs by the cancerous cells with absorption of drugs by normal tissue in accordance with aspects of the innovation.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding of the subject innovation. It may be evident, however, that the innovation can be practiced without these specific details. In other instances, well-known structures and devices are shown in block diagram form in order to facilitate describing the innovation.

While specific characteristics are described herein (e.g., thickness, orientation, configuration, etc.), it is to be understood that the features, functions and benefits of the innovation can employ characteristics that vary from those described herein. These alternatives are to be included within the scope of the innovation and claims appended hereto.

While, for purposes of simplicity of explanation, the one or more methodologies shown herein, e.g., in the form of a flow chart, are shown and described as a series of acts, it is to be understood and appreciated that the subject innovation is not limited by the order of acts, as some acts may, in accordance with the innovation, occur in a different order and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with the innovation.

A unique endoscopic platform for disease demarcation, treatment planning, optimized light delivery and therapy response monitoring is disclosed herein. The platform is based on an endoscopic imaging system that allows both quantitative imaging and adaptive beam-shaping for light-based therapy of diseases. The innovation demarcates and treats diseases, thereby improving survival rates of patients.

This single platform quantifies spatial maps of optical parameters and drug fluorescence concentration for treatment planning and light dose optimization as well as tissue oxygen saturation, hemoglobin concentration and blood flow distributions for monitoring light-based therapies. Currently most clinical endoscopes use white light illumination and reflected light visualization by eye or by charge-coupled device (CCD) camera for visual guidance. In addition some clinical endoscopes are used for direct fluorescence signal intensity imaging. The endoscopic system disclosed herein has three main functions: 1) imaging with enhanced contrast, 2) therapeutic light delivery, 3) monitoring the therapy response for predicting or optimizing the outcome with a feedback loop/response. The imaging part involves both quantitative reflectance and fluorescence imaging. Quantitative refers to extracting the actual chromophore and fluorophore concentrations rather than raw intensity counts.

Standard light dosimetry is based on the prescribed incident dose, which does not take into account reflected and scattered light in the lesion. For optimal light dosimetry with complete cell killing and minimal damage to normal tissue, an appropriate treatment field has to be outlined and sufficient dose delivered. The innovative endoscopic system can perform reflectance and fluorescence measurements by using a single light source and two CCD cameras working in parallel for fast data acquisition during phototherapy without treatment light interruptions. Reflectance measurements can quantify tissue absorption and scattering, which are crucial for accurate light dosimetry in tissue. Reflectance measurements also allow quantification of tissue blood oxygenation and blood volume which are useful for assessing the therapeutic response. The endoscopic system can also assess absolute drug concentration for improved contrast during treatment field planning, and drug dose (concentration) for accurate light dosimetry, and can quantify phototherapy induced physiologic changes (e.g. changes in oxygenation) more accurately and thus inform about the therapy response. Drug (e.g., photosensitizers for photodynamic therapy or doxorubicin used in chemotherapy) fluorescence measurements allow quantification of fluorescence concentration. The endoscopic system determines the optimal light dose according to light attenuation and drug concentration distribution on an individual basis. The endoscopic system allows for real-time treatment light adjustment in the same platform for tumors of any size and shape by adjusting light intensity according to drug concentration so that optimized light delivery can be increased for the cases of cells that have low amount of drug concentrations.

The endoscopic system combines guidance of phototherapy treatment field, the optimization of the treatment light and therapy monitoring in the same platform. The endoscopic system introduces a single platform that allows both imaging and treatment light delivery according to any tumor size and shape that is locally identified by the spatial distribution of drug fluorescence concentration rather than the prescribed fixed dose and shape. The endoscopic system allows near real-time dose optimization on an individual basis by quantifying the spatial distribution of drug concentration and adapting the treatment light dose accordingly. The endoscopic system can be applied to phototherapy optimization of many sites of internal cavities including lung, oral, ovarian, esophagus and colon as well as more accessible sites such as skin, brain and breast.

The endoscopic system provides a method of delivering light using feedback control, wherein dose metrics (drug concentration, tissue blood flow and oxygenation) are monitored and the delivery of treatment light is adjusted accordingly to optimize treatment response.

The endoscopic system allows light delivery concurrently during reflectance and fluorescence measurements. Reflectance measurements provide optical properties (absorption and scattering) of tissue, which in turn allows a) light intensity distribution throughout tissue volume, which is related to light dosimetry in tissue, and b) tissue blood oxygen saturation and blood volume, which is related to treatment response. Fluorescence measurements allow quantification of drug concentration during therapy, which can lead to optimal drug light and drug delivery to target areas.

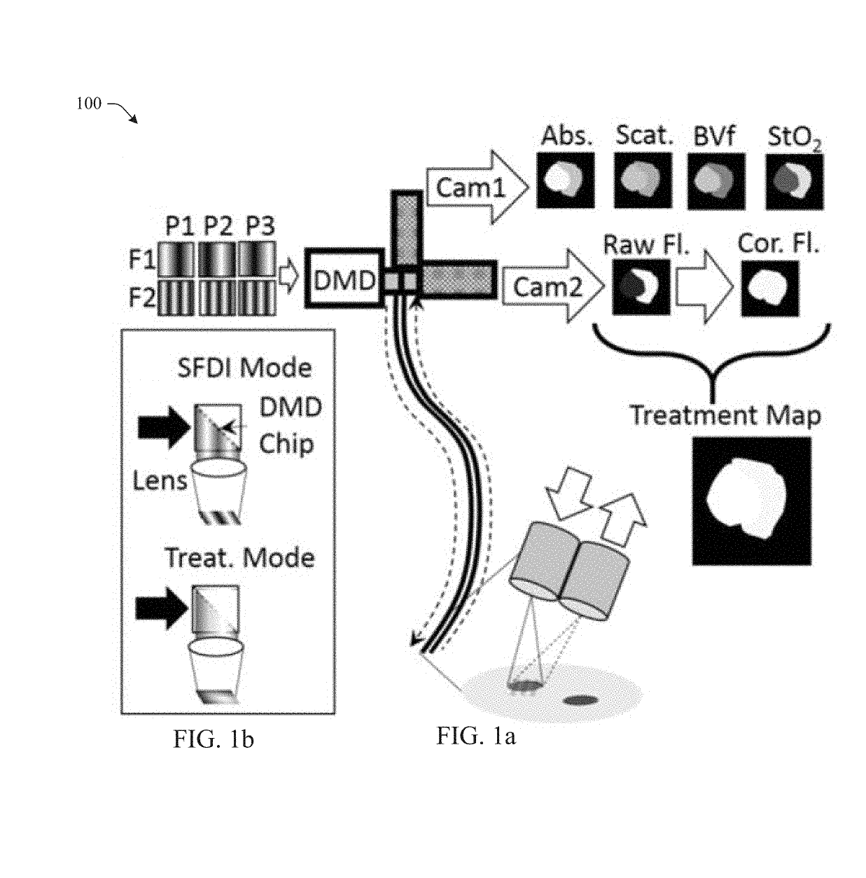

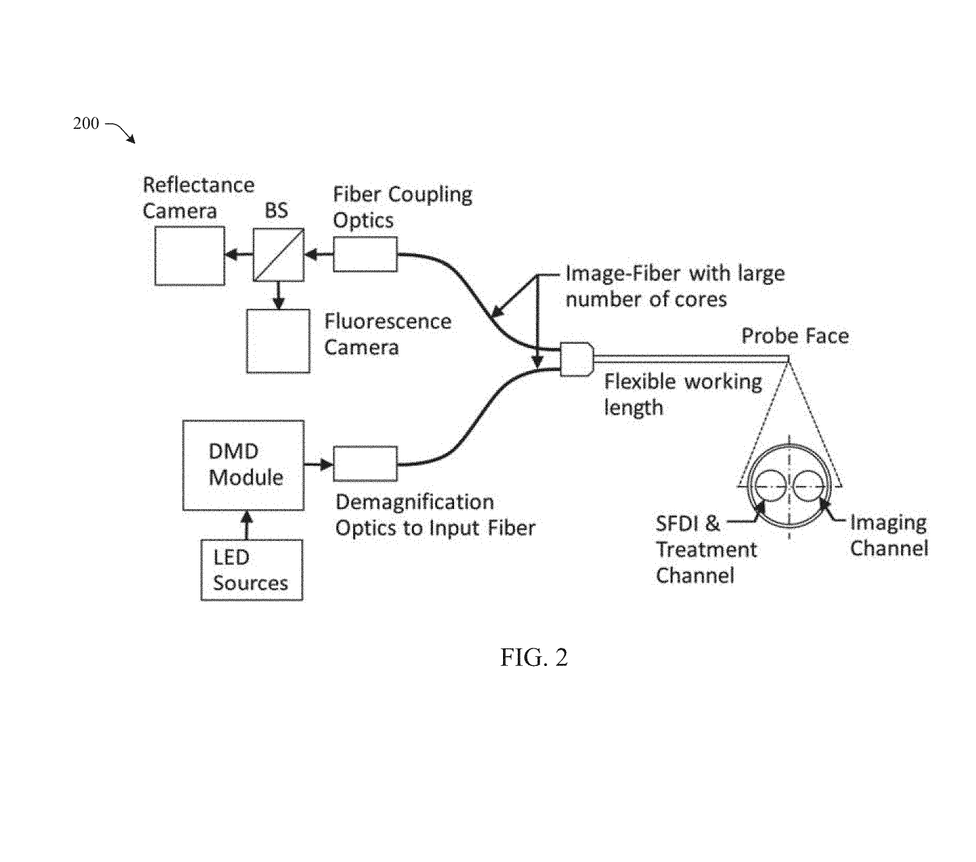

Referring now to the drawings, FIG. 1a illustrates an endoscopic system 100 that combines an imaging and treatment system in the same instrument. The innovative endoscopic system 100 and approach implements structured illumination scheme for quantitative endoscopic fluorescence imaging and treatment. Sine wave patterns are generated and projected onto target using the custom fiberscope. Reflected fluorescence images are corrected for the effects of optical properties to extract maps of true fluorescence. These maps are used to generate a treatment mask which is delivered using the same instrument. FIG. 1b illustrates a representative diagram showing light beam shaping and intensity delivering according to target disease shape.

The endoscopic system 100 includes a high-power, multi-wavelength LED array with bandpass filters at the desired wavelength. The light is directed onto a digital micro-mirror device (DMD), which generates the appropriate spatial frequency pattern. Reflectance and fluorescence images are separated by a dichroic mirror. The endoscopic system 100 can provide high contrast for improved disease demarcation and perform light-based therapies. Light delivery can be adapted according to the sizes and shapes of disease that will be locally identified by the spatial distribution of drug concentration.

More specifically, digital micro-mirror device (DMD) projects spatially modulated light (structured illumination) onto tissue and the measured spatially modulated tissue response function contains information of optical absorption, scattering and fluorescence. Multiple frequencies of a sine wave pattern are generated at three phases (0°, 120° and 240°). The frequencies vary from low (FIG. 1a, top row) to high (FIG. 1a, bottom row). The endoscope is inserted into internal cavity and the patterns are projected down the first channel of the scope and onto the target region (channel follows dotted arrow exiting the instrument). Reflected light is collected in the second channel and relayed to two cameras for reflectance and fluorescence measurements. Once all the images are analyzed, the absorption (μₐ) and scattering (μₛ) properties of the tissue are extracted. By determining μₐ and μₛ at the excitation and emission wavelengths, one can compensate the raw fluorescence attenuation for improved visualization of disease. Then, treatment light intensity and shape will be adjusted according to target shape and fluorescence distribution using the same DMD module with a novel adaptive illumination scheme. The treatment light dose can also be controlled to release a predicted amount of chemo drug in a time specific manner. The illumination scheme can adjust for any shape and individual DMD pixels can project variable light intensity distribution for optimized light delivery. In treatment light delivery mode, the system projects an optimized treatment field onto the target disease while normal tissue is spared.

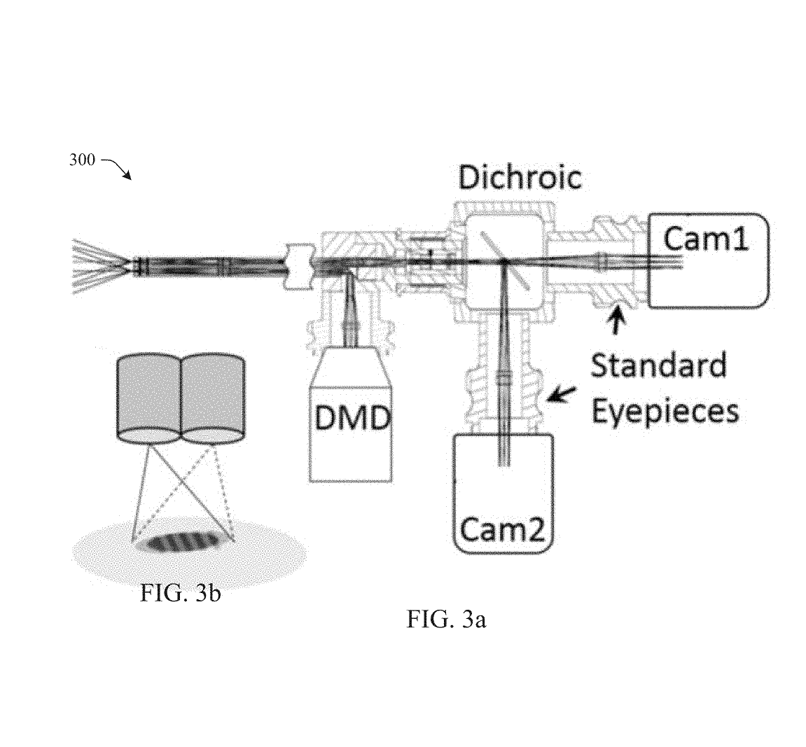

FIG. 2 illustrates a diagram of the flexible endoscope (fiberscope) version 200. The dual-channel fiber based approach allows light projection and imaging to be performed in separate, dedicated channels, which is critical to ensure that any specular reflection of the illumination channel will not interfere with the detection channel. The objective lenses in the tip of the fiberscope provide overlapping field of views (FOVs) of illumination and detection. Compact LEDs can be used for measuring excitation/fluorescence of the target compounds such as porphyrins and chemo drugs like doxorubicin. An LED controller sequentially selects the desired excitation wavelength and light is directed with a liquid light guide to a compact DMD module, where it is spatially modulated to produce the desired pattern. Structured light from the modulator may be focused onto imaging fiber and relayed to the tip. The reflected and fluorescence light collected by the second (detection-channel) imaging fiber is relayed and focused through a dichroic mirror onto one of two cameras depending on the wavelength. Both the projection and imaging fibers (Fujikura Image Fiber) contain 30,000 picture elements in a 650 μm diameter bundle, ensuring high resolution while maintaining flexibility.

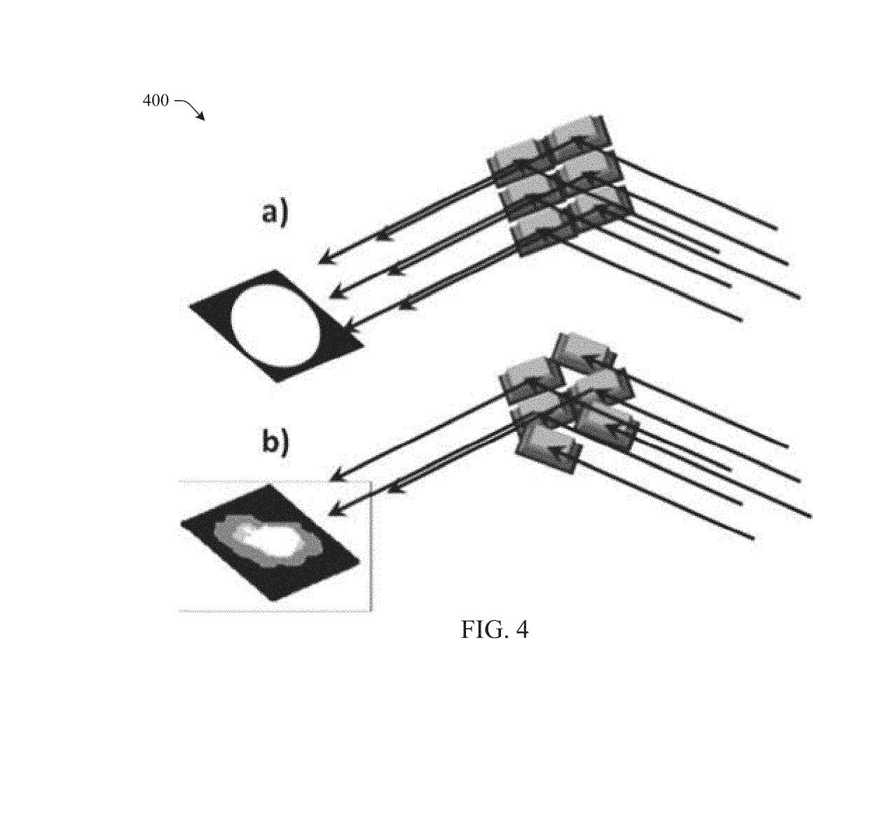

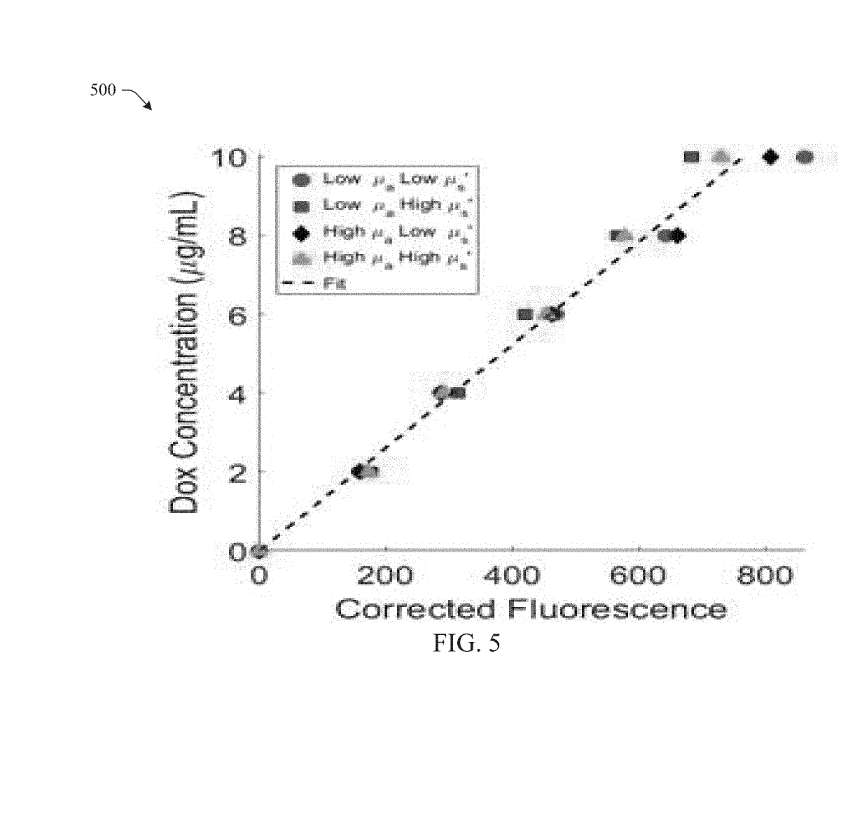

Referring to FIG. 3a, a laparoscopic (rigid endoscope) version 300 can be implemented with a similar approach. This system will be based on a dual channel laparoscope design, utilizing custom rod lenses, right angle prism and eyepieces. This dual channel laparoscope approach will allow light projection and detection imaging to be performed in separate, dedicated channels. This design feature is critical to ensure that any specular reflection of the illumination source from the rod lens surfaces will not interfere with the detected images from tissue. This design also has a compact frame (approximately 1 cm scope outer diameter), which is useful for imaging internal cavities.

FIG. 3a also illustrates a model of the complete instrument with the dual channel laparoscope, light modulator and two compact CCD cameras for reflectance and fluorescence measurements. Structured light from the modulator is directed into the projection side of the dual channel laparoscope. A right angle prism reflects light down the rod lens and the optics at the tip form an image of the desired pattern on the target surface. The reflected and fluorescence light is collected by the “detection side”, which will image the same FOV of the “projection side”. Structured light in this channel is relayed to a dichroic mirror, which directs the light shorter than approximately 700 nm to the first CCD camera for reflectance measurements while longer wavelength light, including fluorescence, is passed through to the second CCD camera for fluorescence measurements.

FIG. 3b illustrates the optics in the tip of the laparoscope, indicating the overlapping FOVs of illumination and detection.

Most clinical endoscopes are utilized for visualization during screening without any quantitative imaging capabilities. Several fluorescence endoscopes have been developed to date but they mainly measure the relative fluorescence intensity not the concentration of the fluorescence drug. These approaches are semi-quantitative; obtained results can vary and depending on the operator, methods, and instrument themselves.

The innovative endoscopic system 100 disclosed herein is based on quantitative reflectance and fluorescence imaging. This device is capable of three main functions: 1) reflectance-related quantitative measures, such as optical absorption and scattering, tissue oxygen saturation and blood flow, and 2) quantitative fluorescence measures such as drug concentration, 3) provide adaptive light treatment illumination scheme. The first two are related to imaging (diagnostic), and the third is related to therapy. Thus, the device is called theranostic light endoscope.

One advantage of the endoscopic system 100 is that the same DMD chip is used for imaging (quantification of optical parameters and drug concentration) as well as light treatment. This allows each pixel from the images to correspond one-to-one with the treatment light. With this setup, fluence rate can be controlled pixel by pixel allowing for precise beam-shaping and fluence rate control.

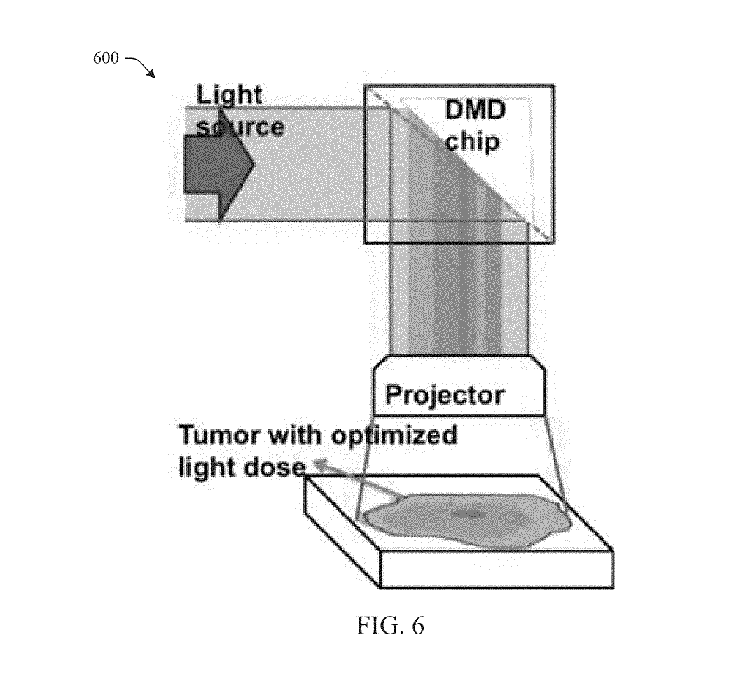

FIGS. 4a and 4b illustrate a procedure 400 where in imaging mode, the light source shines onto DMD mirror and is projected onto tissue. The camera system images the tumor. In treatment mode the light source, DMD mirror, and the tissue project optimized treatment field onto tumor. More specifically, FIGS. 4a and 4b shows the details of the DMD chip, which may contain millions of small mirrors, that can either reflect incoming light (“on”) or deflect it away (“off”). FIG. 4a illustrates all the mirrors operating in the “on” position giving a uniform illumination. FIG. 4b shows some mirrors being turned to the “off” position to produce an optimized illumination that varies in both shape and intensity (fluence rate). The instrument allows imaging optical parameters and fluorescence while directing light treatment by utilizing the same DMD mirror. In imaging mode, the light source shines onto DMD mirror and is projected onto tissue. A high-resolution dual CCD camera system performs the image acquisition. In treatment mode, the system projects an optimized treatment field on to the target. The illumination scheme can adjust for any shape and individual DMD pixel can project variable light intensity distribution for optimized light dosimetry rather than predetermined constant intensity.

The endoscopic system 100 provides fluorescence concentration measurements during therapy without requiring interruption, thereby providing quantitative feedback for adaptive light illumination, which is expected to lead to improved treatment outcome. The endoscopic system 100 provides reflectance measurements during therapy without requiring interruption, allowing more accurate assessment of the kinetics of therapy related parameters (such as tissue blood oxygenation) that are indicative of the treatment response.

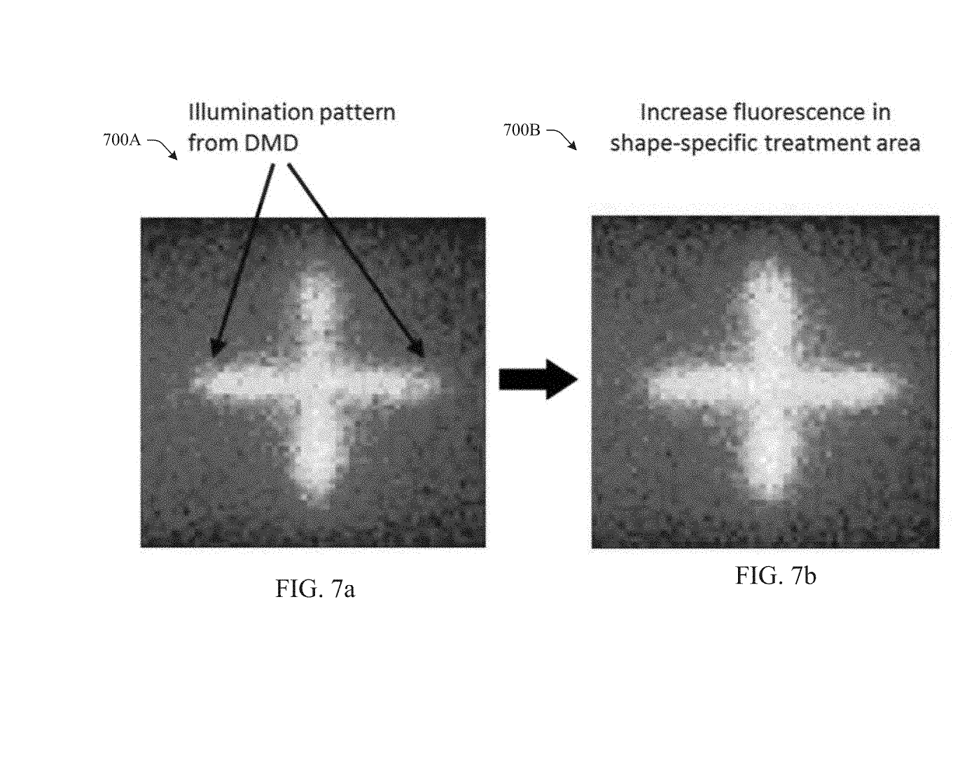

FIG. 5 is a plot 500 illustrating that optical attenuation corrected fluorescence is linear with doxorubicin concentration. Detection sensitivity with minimum concentration of (˜10 ng/mL) in accordance with an aspect of the innovation. The endoscopic system 100 utilizes a special camera, an electron multiplying CCD camera (EMCCD) for improved sensitivity for the low light and low drug concentration cases. The EMCCD provides high sensitivity for drug fluorescence imaging by utilizing the EM gain to reduce the read-out noise significantly. In total, the endoscopic system 100 can achieve unprecedented imaging sensitivity by using structured illumination with efficient filtering, background subtraction and electron-multiplying gain of the CCD camera, a more than 10-fold increased sensitivity has been achieved. The corrected fluorescence signals showed a very good correlation with the drug (doxorubicin) concentration (approximately r²=0.99).

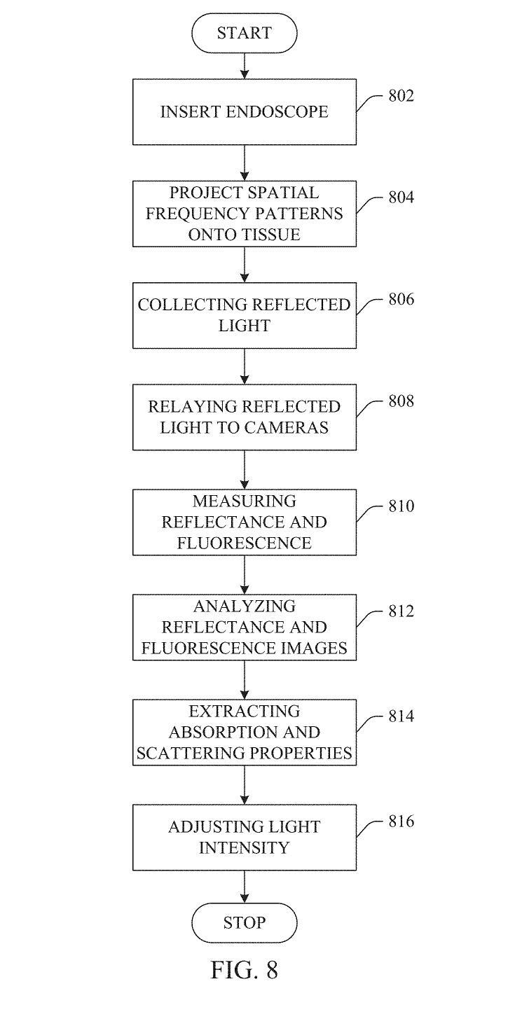

FIG. 6 illustrates a system 600 showing that light can be delivered in any shape and different intensity according to the disease location and shape in accordance with an aspect of the innovation. The treatment field is adjusted to any shape according to the drug distribution, rather than fixed circular shape. The treatment region is optimized according the fluorescence distribution so that diseased site is treated optimally while normal tissue is spared from the side effects. The apparatus can deliver optimal light intensity according to target. In this particular case, several DMD pixels at the center area are focused on the disease to deliver optimized light dose while reducing the light dose at the surrounding normal tissue to minimize the side effects.

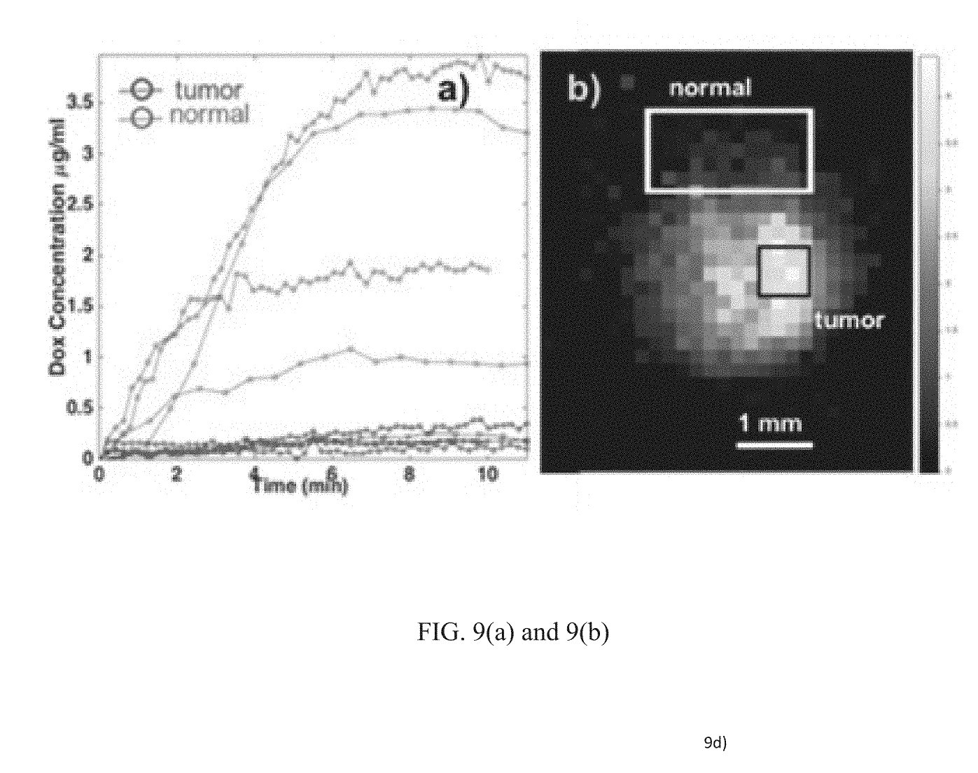

FIGS. 7a and 7b illustrate an optimized light illumination projections 700A, 700B. The endoscopic system can adapt the illumination scheme according to the fluorescence map obtained by the imaging mode. For example, as shown in FIG. 7A, light can be projected with any shape (e.g., in the shape of a plus sign “+”). As shown in FIG. 7b the light intensity can be adapted that can be monitored by fluorescence intensity (increased in the “+” region) rather than having a fixed value.

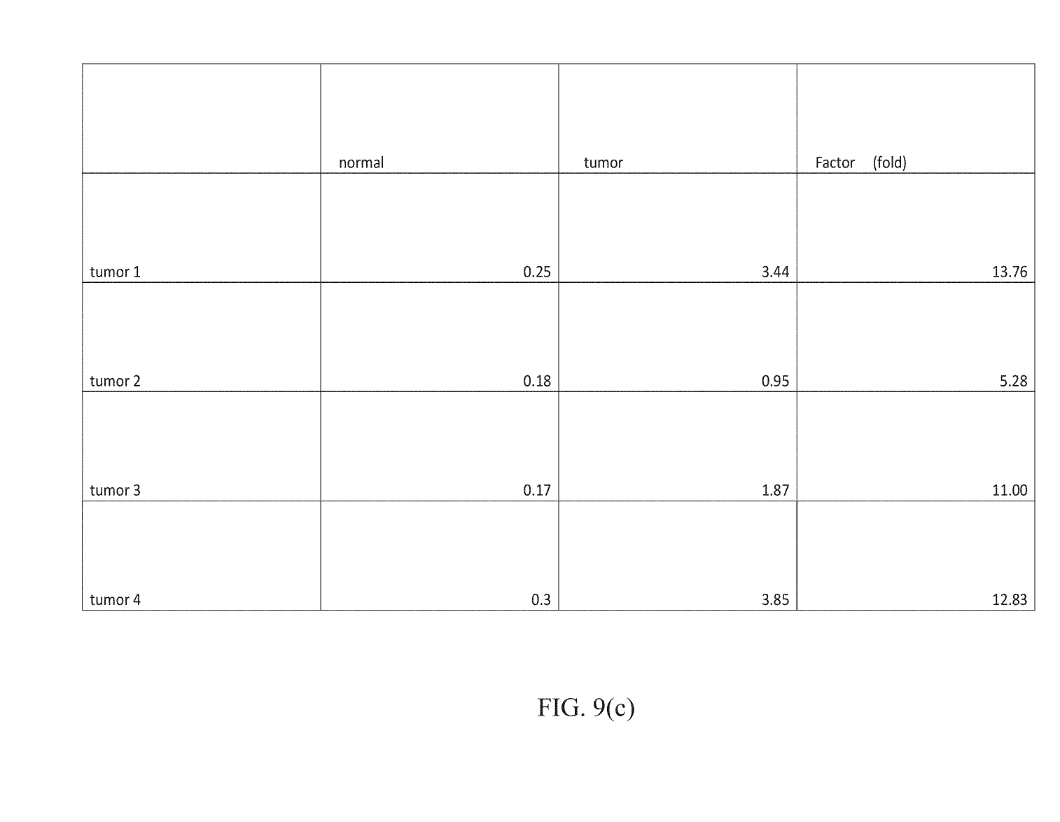

Referring to FIG. 8, a method of performing phototherapy will be described. At 802, and endoscope is inserted into an internal cavity of a patient. At 804, spatial frequency patterns are projected through a first channel of an endoscope onto tissue. At 806, reflected light through a second channel of the endoscope is collected. At 808, the reflected light is relayed to a pair of cameras. At 810, a reflectance in one of the pair of cameras and a fluorescence in the other of the pair of cameras is measured. At 812, the reflectance and fluorescence images are analyzed. At 814, absorption and scattering properties of the tissue are extracted. At 816, a treatment light intensity and shape is adjusted based on the absorption and scattering properties.

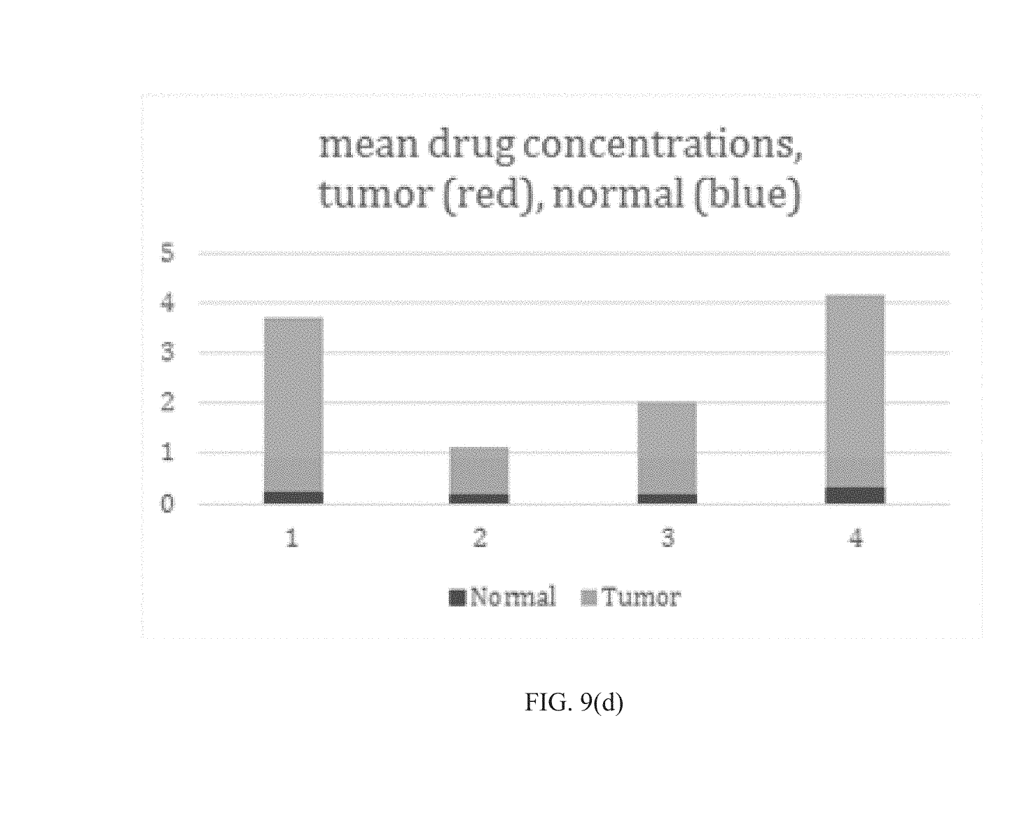

FIG. 9a shows Dox release kinetics from various mouse tumors and corresponding surrounding normal tissue, obtained with our endoscopy device. Compared to normal tissue, tumor shows heterogeneity with respect to release kinetics in the release with respect to individual tumors or tissue. This result implies there is a need for individualized light dosimetry, such that each tumor will require (or otherwise benefit from) different light dose (and illumination pattern according to their shapes) for complete release.

FIG. 9b similarly shows that drug release on a tumor is greater than drug release on the surrounding tissue. It is to be appreciated that, this is due to targeted illumination through the endoscope.

FIG. 9c shows an example table contrasting drug concentrations in tumors and surrounding normal tissue.

FIG. 9d shows an example plot contrasting mean drug concentrations in tumors and surrounding tissue. As the plot clearly shows, the endoscopy device can obtain more than 10-fold drug release at a tumor than in surrounding, normal tissue (e.g., in mice).

In another potential embodiment an endoscopy device, such as is described above, may be applied to neuroscience rather than cancer. Such an endoscope could image neuronal cells (imaging) and then trigger the cells with laser light (neuromodulation). The triggering mechanism for this particular embodiment is based on laser-induced heat that creates changes in action potentials resulting in cell triggering.

Unique characteristic of the endoscopic system is that it can provide feedback for treatment light adjustment by using drug concentration distribution, rather than delivering a fixed light intensity. This provides optimal light dose for complete cell kill or optimal drug delivery for improved therapy outcome. As mentioned and as shown in FIG. 7b, the intensity could be increased, which could be utilized for achieving at least minimal threshold dose to kill the cell, or for the case of drug release, where one needs minimum threshold light intensity to start the drug release process.

The innovative endoscopic system allows quantitative fluorescence imagining in addition, to white-light visualization. The system can quantify fluorescence concentration. In addition, the system can quantify optical parameters of absorption and scattering and oxy deoxy-hemoglobin concentrations, thus tissue blood oxygen saturation and blood volume.

The system can be used in imaging and treating of micrometastasis in intraperitoneal cavity, imaging the brain during surgery for surgical guidance or any drug based intervention as well as in oral, lung, esophageal, cervix cancer detection and treatment. Diagnosis and treatment of gastrointestinal tract, pancreas, urinary bladder, breast. Diagnosis of vulnerable and non-vulnerable atherosclerotic plaques, Alzheimer Disease.

What has been described above includes examples of the innovation. It is, of course, not possible to describe every conceivable combination of components or methodologies for purposes of describing the subject innovation, but one of ordinary skill in the art may recognize that many further combinations and permutations of the innovation are possible. Accordingly, the innovation is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

The above embodiments have been described with brevity (e.g., in terms of modules) in light of what is well known in the art. However, it should be understood that some of the inventive functionality, features and benefits recited herein may be carried out in connection with the use of computers, processors, software, computer logic, hardware and other implements known to one of ordinary skill in the art as necessary.

What is claimed is:

1. An endoscopic imaging system comprising:
an endoscope having a first channel and a second channel;
a high-power, multi-wavelength LED array;
a digital micro-mirror device that receives light directed from the high-power, multi-wavelength LED array and generates spatial frequency patterns; and
a dichroic mirror that separates reflectance and fluorescence images.

2. The endoscopic imaging system of claim 1, wherein the digital micro-mirror device projects spatially modulated light onto tissue, wherein the measured spatially modulated tissue response function contains information of optical absorption, scattering and fluorescence

3. The endoscopic imaging system of claim 1, wherein spatial frequency patterns are projected down the first channel.

4. The endoscopic imaging system of claim 3, wherein reflected light is collected in the second channel and relayed to two cameras for reflectance and fluorescence measurements.

5. The endoscopic imaging system of claim 1, wherein the endoscope is a laparoscope design utilizing custom rod lenses, right angle prism and eyepieces.

6. The endoscopic imaging system of claim 5, wherein a right angle prism reflects light down the rod lens and optics at the tip form an image of the desired pattern on a target surface.

7. The endoscopic imaging system of claim 6, wherein the reflected and fluorescence light is collected the “detection side” which will image the same file of vision as the “projection side”.

8. The endoscopic imaging system of claim 7, wherein structured light in this channel is relayed to the dichroic mirror, wherein the dichroic mirror is operative to direct light shorter than approximately 700 nm to a first CCD camera for reflectance measurements while longer wavelength light, including fluorescence, is passed through to a second CCD camera for fluorescence measurements.

9. The endoscopic imaging system of claim 8, wherein several DMD pixels at the center area of the apparatus are focused on the disease to deliver optimized light dose while reducing the light dose at surrounding normal tissue.

10. The endoscopic imaging system of claim 7, wherein a treatment field may be adjusted to any shape and different intensity according drug distribution, rather than a fixed circular shape.

11. The endoscopic imaging system of claim 7, wherein the treatment filed is optimized according to fluorescence distribution so that a diseased site is treated optimally while normal tissue is spared treatment, and thus side effects.

12. The endoscopic imaging system of claim 1, wherein the system is operative to adapt the illumination scheme according to a fluorescence map obtained by the imaging mode.

13. The endoscopic imaging system of claim 1, wherein light intensity can be adapted rather than having a fixed value.

14. A method of phototherapy comprising:
inserting an endoscope into an internal cavity;
projecting spatial frequency patterns through a first channel of the endoscope onto tissue;
collecting reflected light through a second channel of the endoscope;
relaying the reflected light to a pair of cameras;
measuring a reflectance in one of the pair of cameras and a fluorescence in the other of the pair of cameras;
analyzing the reflectance and fluorescence images;
extracting absorption and scattering properties of the tissue; and
adjusting treatment light intensity and shape based on the absorption and scattering properties.

15. The method of claim 5, wherein the absorption and scattering properties are determined at an excitation and emission wavelength.

16. An endoscopic imaging apparatus, the apparatus comprising:
first and second dedicated channels comprised of flexible fiber, wherein the first channel is dedicated to light projection and the other is dedicated to imaging; and
objective lenses in the tip of the endoscope that provide overlapping fields of view for illumination and detection.

17. The endoscopic imaging apparatus of claim 16, further comprising compact LEDs operative to measure fluorescence of target compounds such as porphhyrins and chemotherapy drugs.

18. The endoscopic imaging apparatus of claim 16, further comprising an LED controller operative to sequentially select the desired excitation wavelength and light directed with a liquid light guide to a compact DMD module, where it is spatially modulated to produce a desired pattern.

19. The endoscopic imaging apparatus of claim 16, wherein structured light from a modulator may be focused onto imaging fiber and relayed to the tip.

20. The endoscopic imaging apparatus of claim 16, wherein the reflected and fluorescence light collected by the second (detection-channel) imaging fiber is relayed and focused through a dichroic mirror onto one of two cameras depending on the wavelength.
